(19) United States
(12) Patent Application Publication
(10) Pub. No.: US 2011/0269849 A1
(43) Pub. Date: Nov. 3, 2011

(54) Emulsions and Methods for the Preparation Thereof, and Methods for Improving Oxidative Stability of Lipids

(76) Inventor: Yuan Yao, West Lafayette, IN (US)

(21) Appl. No.: 12/953,215

(22) Filed: Nov. 23, 2010

Related U.S. Application Data
(60) Provisional application No. 61/330,483, filed on May 3, 2010.

(51) Int. Cl. A23D 7/06; A23D 9/06; C09K 15/20; A23L 1/035; A61K 47/42; A01N 25/22

(52) U.S. Cl. 514/773; 252/403; 426/610; 426/602

(57) ABSTRACT

An emulsion includes a lipid, an emulsifier, and ε-polylysine. A method for improving oxidative stability of a lipid includes forming an emulsion that includes the lipid, an emulsifier, and ε-polylysine, such that the emulsion further includes a complex layer at an oil-water interface configured to provide a physical and/or electrostatic barrier against oxidation of the lipid. A method for preparing an emulsion includes combining a lipid, an emulsifier, and ε-polylysine in a mixture; and homogenizing the mixture to provide an emulsion.

### RELATED APPLICATIONS

This application claims the benefit of U.S. Provisional Application No. 61/330,483, filed May 3, 2010, the entire contents of which are hereby incorporated by reference.

### TECHNICAL FIELD

The present teachings relate generally to emulsions and to methods for their preparation and use.

### BACKGROUND

The oxidation of lipid compounds in food emulsions leads to rancidity and the generation of toxic compounds. Thus, reducing lipid oxidation is desirable for improving the stability of oil-in-water emulsions, such as are widely used in the food, nutraceutical, drug, personal care, and other industries. A variety of methods have been developed to reduce lipid oxidation, such as the addition of antioxidants, the removal of oxygen, and the use of purified food ingredients to reduce transition metal ions. These methods help to control radicals, oxidation catalysts, oxidation intermediates, and secondary lipid oxidation breakdown products. However, these methods typically require high-cost ingredients and/or complicated processes to achieve appreciable improvements in oxidative stability.

### SUMMARY

The scope of the present invention is defined solely by the appended claims, and is not affected to any degree by the statements within this summary.

By way of introduction, a first emulsion in accordance with the present teachings includes a lipid, an emulsifier, and ε-polylysine.

A second emulsion in accordance with the present teachings includes a lipid, an emulsifier selected from the group consisting of anhydride-modified phytoglycogen, anhydride-modified glycogen-type material, anhydride-modified starch, and combinations thereof; and ε-polylysine.

A method for improving oxidative stability of a lipid in accordance with the present teachings includes forming an emulsion that includes the lipid, an emulsifier, and ε-polylysine. The emulsion further includes a complex layer at an oil-water interface configured to provide a physical and/or electrostatic barrier against oxidation of the lipid.

A method for preparing an emulsion in accordance with the present teachings includes combining a lipid, an emulsifier, and ε-polylysine in a mixture; and homogenizing the mixture to provide an emulsion. The emulsifier is selected from the group consisting of anhydride-modified phytoglycogen, anhydride-modified glycogen-type material, anhydride-modified starch, and combinations thereof.

### BRIEF DESCRIPTION OF THE DRAWINGS

FIG. 1 shows a two-dimensional schematic of a phytoglycogen nanoparticle (A) and a segment of amylopectin (B).

FIG. 2 shows the chemical structure of ε-poly-L-lysine (a.k.a. ε-polylysine, polylysine, and EPL).

FIG. 3 shows the accumulation of hydroperoxide (A) and thiobarbituric acid reactive substances (TBARS) (B) of fish oil emulsions formed by different types of emulsifier. Each data point is the mean value of 3 measurements with error bar of standard deviation.

FIG. 4 shows the accumulation of hydroperoxide (A) and TBARS (B) of fish oil emulsions formed using phytoglycogen octenyl succinate (PG-OS) and TWEEN 20 in the presence and absence of 0.1% EPL. Each data point is the mean value of 3 measurements with error bar of standard deviation.

FIG. 5 shows the accumulation of hydroperoxide (A) and TBARS (B) of fish oil emulsions formed using waxy cornstarch octenyl succinate (WCS-OS) in the presence and absence of 0.1% EPL. Each data point is the mean value of 3 measurements with error bar of standard deviation.

FIG. 6 shows the accumulation of hydroperoxide (A) and TBARS (B) of fish oil emulsions formed using PG-OS (degree of substitution or DS 0.0146) with EPL added at 0.05, 0.1, and 0.2% levels. Each data point is the mean value of 3 measurements with error bar of standard deviation.

FIG. 7 shows the average particle size of fish oil emulsions formed using PG-OS, WCS-OS, and TWEEN 20. Each data point is the mean value of 3 measurements with error bar of standard deviation.

FIG. 8 shows the average particle size of fish oil emulsions formed using PG-OS and TWEEN 20 in the presence and absence of 0.1% EPL. Each data point is the mean value of 3 measurements with error bar of standard deviation.

FIG. 9 shows the average particle size of fish oil emulsions formed using WCS-OS in the presence and absence of 0.1% EPL. Each data point is the mean value of 3 measurements with error bar of standard deviation.

FIG. 10 shows the zeta-potential of aqueous dispersions and emulsions containing both PG-OS (DS 0.0146) and EPL. Each data point is the mean value of 3 measurements with error bar of standard deviation.

FIG. 11 shows a schematic of distributions of PG-OS and EPL in aqueous dispersion (A), emulsion with EPL added after emulsification (B), and emulsion with EPL added before emulsification (C). The complex layer comprising both PG-OS nanoparticles and EPL molecules is highlighted by the ringed area in (C).

### DETAILED DESCRIPTION

Oil-in-water emulsions that contain lipid, emulsifier, and ε-poly-L-lysine (a.k.a. ε-polylysine, polylysine, and EPL) have been discovered and are described hereinbelow. Surprisingly and unexpectedly, emulsions in accordance with the present teachings have high lipid oxidative stability as compared to conventional emulsions—an improvement that is affected by the type of emulsifier used and by the amount of EPL added. Without wishing to be bound by a particular theory or to in any way limit the scope of the appended claims or their equivalents, it is presently believed that a combined use of an emulsifier and EPL forms a complex layer at the oil-water interface, which provides a physical and/or electrostatic barrier against pro-oxidative compounds.

It has further been found that the presence of EPL (at appropriate levels) does not affect the physical stability of some emulsions. Applications of emulsions in accordance with the present teachings are unrestricted and include but are not limited to their use in the food and beverage industry wherein very low lipid oxidation and rancidity are particularly sought after (e.g., in food or beverages containing nutritional lipid compounds, such as omega-3 fatty acids), as well as in the cosmetics, pharmaceutical, chemical, agricultural (e.g., pesticides), microbiology, and polymer industries to name but a few. In some embodiments, the emulsions are prepared by mixing a lipid, emulsifier, EPL, and water (or buffer), and then applying homogenization for effective emulsification. Representative lipids for use in accordance with the present teachings include all manner of lipophilic compounds including but not limited to omega-3 fatty acids (e.g., fish oil, docosahexaenoic acid or DHA, eicosapentaenoic acid or EPA, etc.) and ester derivatives thereof, other saturated, unsaturated and/or polyunsaturated fatty acids (e.g., mono-, di-, and triglycerides, phospholipids, etc.) and ester derivatives thereof, and the like, and combinations thereof.

Emulsifiers for use in accordance with the present teachings are unrestricted and include all manner of emulsifiers—particularly though not exclusively food-grade emulsifiers. Representative emulsifiers contemplated for use include but are not limited to amphiphilic proteins, phospholipids, small molecule surfactants, polysorbate surfactants (such as the polyoxyethylene derivative of sorbitan monolaurate known as polysorbate 20 and sold under the tradename TWEEN 20 by Croda International PLC), gum arabic, starch, modified starch (e.g., anhydride-modified starch, such as starch octenyl succinate), phytoglycogen, modified phytoglycogen, glycogen-type materials, modified glycogen-type materials, and the like, and combinations thereof. Modification in the sense used herein refers to chemical modifications (e.g., such as is achieved via reaction with an anhydride), and includes but is not limited to the alkylation and/or carboxylation of one or more hydroxyl moieties of starch, phytoglycogen, and/or glycogen-type materials.

It has further been discovered that modified phytoglycogen and glycogen-type materials have a particularly strong capacity for stabilizing emulsions in accordance with the present teachings. As used herein, the phrase “phytoglycogen or glycogen-type material” refers to dendritic (i.e., highly branched) α-D-glucan and carbohydrate nanoparticles. The term “phytoglycogen” generally refers to material that is derived from plants while the term “glycogen” generally refers to material that is derived from microbials and/or animals.

One such modified phytoglycogen for use in accordance with the present teachings is the amphiphilic dendritic molecule phytoglycogen octenyl succinate (PG-OS). PG-OS is an amphiphilic carbohydrate nanoparticle prepared using octenyl succinate (OS) substitution of phytoglycogen (PG). PG-OS has a dispersed molecular density nearly 20 times that of waxy cornstarch octenyl succinate (WCS-OS). PG-OS with a degree of substitution (DS) of 0.015 and 0.048 was prepared by reacting PG with 3% and 9% of octenyl succinic anhydride, respectively.

In some embodiments, PG-OS and EPL were used to form oil-in-water emulsions with enhanced lipid oxidative stability. Fish oil-in-water emulsions were prepared using PG-OS, WCS-OS, and TWEEN 20, stored at 55° C. for 6 days, and monitored for the accumulation of hydroperoxide and TBARS. The results indicated that PG-OS correlates with high lipid oxidative stability, and that the addition of EPL may further improve the oxidative stability of emulsions. To address the interaction between PG-OS and EPL, zeta-potential was determined for various systems. As further described below, the results indicated a possible formation of an interfacial complex layer comprising both PG-OS and EPL. Without wishing to be bound by a particular theory or to in any way limit the scope of the appended claims or their equivalents, it is presently believed that this complex layer may provide both physical and electrostatic barriers against pro-oxidative compounds.

By way of background, PG is a starch-like α-D glucan present in plants. Different from starch, PG does not show the semi-crystalline structure of starch and is water-dispersible due to its high branch density. The largest source of PG is the kernel of the maize mutant sugary-1 (su1), a primary genotype of commercial sweet corn. The su1 mutation leads to the deficiency of SU1, an isoamylase-like starch debranching enzyme (DBE). In the biosynthesis of starch, starch synthase, starch branching enzyme, and DBE work together to produce starch granules. The primary role of DBE is to trim abnormal branches that inhibit the formation of starch crystals and granules. In the absence of DBE, the highly branched PG is formed to replace starch.

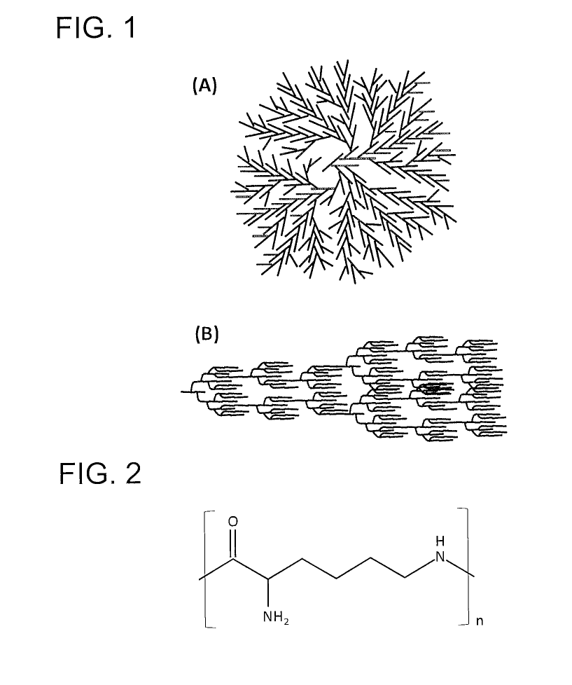

Transmission electron microscope (TEM) and cryo-TEM showed that PG ranges from 30 to 100 nm in particle size and exhibits a spherical shape. The highly branched structure of PG results in its unusually high molecular density in dispersion. In rice, the dispersed molecular density of PG is over 10 times that of starch. As shown in FIG. 1, each PG particle contains hundreds or thousands of glucan chains forming a highly packed structure. Without wishing to be bound by a particular theory or to in any way limit the scope of the appended claims or their equivalents, it is presently believed that that the spherical PG particle grows from the non-reducing ends on the surface by periodic branching and elongation of chains. As further shown in FIG. 1, there is no long chain in PG that connects individual clusters as in the case of an amylopectin molecule, which suggests a fundamental structural difference between PG and amylopectin.

Emulsifiers for use in accordance with the present teachings are not limited to modified PG and glycogen-type materials. By way of example, other representative materials include but are not limited to starch octenyl succinate, which offers both steric hindrance and electrostatic repelling among oil droplets, thus leading to high stability of emulsions, and which has shown superior functionality compared with gum arabic for encapsulating flavors and fish oil, especially for improving oxidation stability.

A further representative emulsifier for use in accordance with the present teachings includes waxy cornstarch octenyl succinate (WCS-OS). In some embodiments, WCS-OS with DS of 0.015 and 0.048 was prepared by reacting waxy cornstarch (WCS) with 3% and 9% of octenyl succinic anhydride, respectively. TWEEN 20 (purchased from Sigma) was also used.

ε-Polylysine is a cationic, naturally occurring homo-polyamide of L-lysine produced by Streptomyces albulus, and is an FDA-GRAS food ingredient. In EPL molecules, the amide linkages are formed between the ε-amino and α-carboxyl groups (FIG. 2). EPL is water soluble, biodegradable, edible, and nontoxic, and has been used in a variety of applications such as food preservatives, emulsifying agents, dietary agents, biodegradable fibers, highly water absorbable hydrogels, and other functional agents. Heretofore, there has been no report of using EPL as an antioxidant in either emulsion or non-emulsion systems. Without wishing to be bound by a particular theory or to in any way limit the scope of the appended claims or their equivalents, it is presently believed that EPL improves oxidative stability via an interfacial repelling effect against metal ions.

The following examples and representative procedures illustrate features in accordance with the present teachings, and are provided solely by way of illustration. They are not intended to limit the scope of the appended claims or their equivalents.

Materials and Methods

Waxy cornstarch was a gift from National Starch Food Innovation (Bridgewater, N.J.). Sweet corn Silver Queen (a sugary line) was purchased from Burpee Co. (Warminster, Pa.). 1-Octenyl succinate anhydride (OSA) was a gift from Dixie Chemical Co. (Houston, Tex.). Fish oil from menhaden and TWEEN 20 were purchased from Sigma-Aldrich (St. Louis). ε-Polylysine (25.8% w/w in water) was obtained from Purac America (Lincolnshire, Ill.).

Extraction of PG

Sweet corn kernels were ground into grits and then mixed with 4 to 6 weights of deionized water. The suspension was homogenized using a high-speed blender (Waring Laboratory, Torrington, Conn.) and then centrifuged at 5500×g for 20 minutes. The supernatant was collected while the solid was further extracted twice using deionized water. Thereafter, the supernatant at each batch was combined and passed through a 270-mesh sieve. The liquid was then added to 3 volumes of ethanol to precipitate the polysaccharide. After centrifugation and decanting the supernatant, the precipitate was further dispersed using 3 volumes of ethanol and centrifuged to dehydrate. The suspension after the last ethanol addition was filtered to remove excess liquid. The solid material was placed in a fume hood to remove residual ethanol. The powder collected was the PG material used for further treatments.

General Procedure for Substitution Using OSA

To the suspension of waxy corn starch (20% w/w) and dispersion of PG (20%, w/w), 1-octenyl succinate anhydride was gradually added over 2 hours at the levels of 3 and 9% based on the dry weight of glucans. The pH was maintained between 8.5 and 9.0 using 2% NaOH. The reaction was conducted at room temperature (22° C.) and terminated after 24 hours by reducing the pH to 6.5 using 2% HCl. To collect substituted glucans, three volumes of ethanol were added to the reaction mixture. The precipitated materials were collected and further dehydrated using 3 cycles of ethanol suspension-centrifugation. The solid collected after filtration was placed in a fume hood to remove residual ethanol to prepare dry powder of PG-OS.

Degree of substitution of PG-OS and WCS-OS was determined using a method from the Joint FAO/WHO Expert Committee on Food Additives with modifications. The glucan sample (0.5 g) was acidified with 3 mL of HCl (2.5 M) for 30 minutes. To each mixture, 10 mL of 90% isopropanol (v/v) was added, followed by centrifugation at 3000×g for 10 minutes and supernatant decanting. To the precipitate, an additional 10 mL of 90% isopropanol was added to re-suspend the glucan solid and then centrifugation was conducted. This procedure was repeated until the test of chloride ions using AgNO₃ showed negative. To test for chloride ions, one drop of 0.1M AgNO₃ was added to the pooled supernatant to observe a white haze of AgCl. Once no noticeable AgCl haze was observed, 30 mL of deionized water was added to the glucan precipitate. The mixture was heated in a boiling water bath for 30 minutes, and titrated using 0.01M NaOH. The DS was calculated by DS=162A/(1000−210A), where A (mmol/g) is the molar amount of octenyl succinate groups in one gram of derivative, and 162 and 210 are the molecular weights of the anhydro glucosyl unit and the octenyl succinate group, respectively. The value of A was calculated using A=[(V−V₀)×0.01]/0.5, where V (mL) is the volume of NaOH solution consumed by the octenyl succinate derivative, V₀ (mL) is the volume of NaOH consumed by native PG or starch, 0.5 is the weight of material in grams, and 0.01 is the molar concentration of NaOH.

Dispersed Molecular Density

Weight-average molecular weight (M_{w}), z-average radius of gyration (R_{z}) of PG, WCS, and their octenyl succinate derivatives were determined using an HPSEC-MALLS-RI system (Wyatt Technology, Santa Barbara, Calif.) using two connected columns (PL Aquagel-OH 40 and 60, Polymer Laboratories, Varian Inc.) with a guard column. The flow rate was 1.0 mL/min using deionized water (pH 6.8, containing 0.02% NaN₃) as the mobile phase. Astra software (Version 5.3.4.10, Wyatt Technology) was used to determine M_{w} and R_{z}. The dispersed molecular density (ρ, g/mol·nm³) was calculated as ρ=M_{w}/R_{z}³. ANOVA was conducted using Minitab 15 (Minitab Inc, State College, Pa.), and Tukey test were utilized with a significant F test (P 0.05).

Emulsion Preparation

For PG-OS and WCS-OS materials, 1 g (dry base) of each was dispersed in 0.02 M NaAc buffer to make 20 g dispersion. WCS-OS requires heating in a boiling water bath for 20 minutes for full dispersion. For TWEEN 20, 0.1 g was dissolved in buffer to make a 20-g solution. For groups added with EPL, 77.6 μL of 25.8% EPL solution was added to the mixture. This made the concentration of EPL to be 0.10%. In addition, for the emulsions using PG-OS (prepared using 3% OSA) as the emulsifier, 38.8 and 155.2 μL of 25.8% EPL solution was added to the mixture, which made EPL concentration to be 0.05 and 0.20%, respectively. For individual dispersions, the pH was adjusted to 6.0, and 0.50 g fish oil was added. Coarse emulsion of fish oil was prepared using a high-speed mixer (T25 ULTRA-TURRAX, IKA) for 1 minute at 18,000 rpm. The coarse emulsions were further treated using a high-pressure homogenizer (Nano DeBEE).

The use of 5% PG-OS (or WCS-OS) and 0.5% of TWEEN 20 was based on the consideration to provide roughly the equivalent amount of hydrophobic moieties. For 1 g of PG-OS or WCS-OS with DS around 0.015, around 0.09 mmol of hydrophobic groups were provided. By contrast, 0.1 g of TWEEN 20 provided around 0.08 mmol hydrophobic groups. For PG-OS and WCS-OS, multiple hydrophobic groups were grafted on each individual glucan molecule. Therefore, it is highly likely that only a fraction of all hydrophobic groups may get access to the oil-water interface.

Storage and Sampling

Immediately after homogenization, an aliquot from each emulsion was used for the analysis of initial particle size, hydroperoxide, and TBARS. All initial measurements were conducted within 3 hours after homogenization. Meanwhile, 8 mL of each emulsion was transferred into a 50-mL capped tube and placed upright and still in a water circulator at 55° C. in the dark. To monitor the physical and oxidative stability, aliquots were taken after 1, 2, 3, 4, 5, and 6 days of storage.

Measurement of Lipid Hydroperoxide

In general, greater lipid oxidation leads to higher lipid hydroperoxide. To measure lipid hydroperoxide, an aliquot of 0.3 mL from each emulsion sample was added to 1.5 mL of isooctane/2-propanol (3:1 v/v) mixture. The mixture was vortexed for 1 minute before centrifugation for 2 minutes at 3200×g. After a preliminary evaluation of hydroperoxide amount for each sample, a proper amount of aliquot of the organic phase was added to methanol/butanol (2:1 v/v) mixture to make up 3.0 mL. To this mixture, 15 μL of ammonium thiocyanate solution (3.94 M) was added, followed by adding 15 μL ferrous iron solution. The mixture was vortexed and incubated for 20 minutes before measuring the absorbance at 510 nm.

Ferrous iron solution was prepared by: (1) dissolving 2 g FeSO₄.7H₂O in 50 mL deionized water, (2) dissolving 1.6 g BaCl₂ dehydrate in 50 mL water, (3) slowly mixing the solutions of FeSO₄ and BaCl₂, (4) adding 2 mL of 10 N HCl, and (5) collecting the clear solution of FeCl₂ after removing BaSO₄ precipitate. The ferrous iron solution was stored at 4° C. in the dark.

To prepare the ferric ion solution for the standard curve, 0.5 g iron powder was dissolved in 50 mL 10 M HCl and 2 mL 30% H₂O₂ solution was added. The solution was heated in a boiling water bath for 5 minutes. After cooling, the solution was diluted with deionized water to 500 mL. The resulting ferric ion standard solution (1000 μg/mL) was further diluted with 1M HCl to obtain final concentrations of 750, 500, 250 and 125 μg/mL.

Measurement of TBARS

In general, greater lipid oxidation leads to higher TBARS. After a preliminary evaluation of TBARS for each sample, an aliquot of 0.4 mL emulsion was added to 0.8 mL thiobarbituric acid-butylated hydroxytoluene (TBA-BHT) solution. The mixture was vortexed for 30 seconds and heated in a boiling water bath for 15 minutes. Thereafter, the mixture was cooled to room temperature and centrifuged at 8500×g for 10 minutes. The absorbance of supernatant was measured at 532 nm against a malonaldehyde standard curve.

The TBA solution was prepared by mixing 15 g trichoroacetic acid, 0.375 g TBA, 1.76 mL 12 M HCl, and 82.9 mL deionized water. The solution of 2% BHT was prepared by dissolving butylated hydroxytoluene in ethanol. TBA-BHT solution was prepared by slowly adding 3 mL BHT solution into 100 mL TBA solution with stirring.

Standard malonaldehyde (MDA) solution was prepared by dissolving 25 μL of 1,1,3,3 tetraethoxypropane (TEP) in 100 mL water to make a 1.0 μmol/mL stock solution. Right before the test, 1.0 mL stock solution was mixed with 50 mL of 1% (v/v) H₂SO₄ and then incubated for 2 hours at room temperature. The resulting MDA standard was 20 nmol/mL. A series of dilutions using 1% H₂SO₄ were made to obtain 15, 10 and 5 nmol/mL standards.

Measurement of Emulsion Particle Size

The emulsions were diluted using 62.5 volumes of 0.01M NaAc buffer pH 6.0. Thereafter, the particle size was measured at room temperature using Zetasizer Nano (ZS90, Malvern Instruments).

Measurement of Zeta-Potential of Dispersions and Emulsions

To prepare dispersions containing PG-OS and EPL, PG-OS prepared using 3% OSA (with DS of 0.0146) was mixed with EPL in 0.02 M pH 6.0 NaAc buffer. For individual dispersions, the concentration of PG-OS was 5% (w/w), and EPL was 0.025, 0.05, 0.1, 0.2, and 0.4% (w/w). To prepare emulsions containing PG-OS and EPL, two procedures were conducted: (1) EPL was added before emulsification and (2) EPL was added after emulsification. The procedure of emulsification is described above. The amount of PG-OS was 5%, and the amount of EPL was 0.025, 0.05, 0.1, 0.2, and 0.4% for both groups with EPL added either before or after emulsification. In each emulsion, 2.5% (w/w based on buffer) of fish oil was used. To measure zeta-potential, dispersions and emulsions were diluted by 62.5 volumes of 0.01 M pH 6.0 NaAc buffer. The measurement was made using Zetasizer Nano at room temperature.

Preparation of PG-OS and WCS-OS

As shown in Table 1, the DS was slightly affected by the type of substrate: PG or WCS. When OSA was used at 3% of glucan, the DS value was 0.0146 and 0.0152 for PG-OS and WCS-OS, respectively. When OSA was added at 9%, the DS value was 0.0475 for PG-OS and 0.0482 for WCS-OS. The substitution efficiency (SE) for 9% OSA (68-69%) was slightly higher than that for 3% OSA (63-66%). In general, the DS values were equivalent between PG-OS and WCS-OS at either the 3 or 9% OSA levels, which allowed direct comparisons between PG-OS and WCS-OS materials.

TABLE 1
Degree of substitution (DS), substitution efficiency (SE), weight-average
molecular weight (M_{w}), z-average radius of gyration (R_{z}), dispersed molecular
density (ρ), and zeta-potential of PG, WCS, and their derivatives after reacting with
3 and 9% of OSA.

| | OSA, % | | SE, | M_{w}, g/ | | ρ, g/ | Zeta- |
|---|---|---|---|---|---|---|---|
| | of glucan | DS × 100* | % | mol × 10⁻⁷** | R_{z}, nm** | mol · nm³** | potential* |
| PG | 0 (native) | | | 3.11 ± 0.09a | 30.5 ± 0.3a | 1095 ± 63a | −3.1 ± 0.1 |
| | 3 | 1.46 ± 0.03 | 63 | 2.94 ± 0.06a | 30.5 ± 1.2a | 1036 ± 104a | −14.9 ± 0.4 |
| | 9 | 4.75 ± 0.06 | 68 | 3.14 ± 0.06a | 33.7 ± 0.2b | 824 ± 21b | −20.3 ± 0.9 |
| WCS | 0 (native) | | | 2.79 ± 0.05a | 60.5 ± 2.5a | 127 ± 14a | −4.2 ± 0.0 |
| | 3 | 1.52 ± 0.02 | 66 | 1.41 ± 0.35b | 63.5 ± 5.0a | 56 ± 13b | −12.0 ± 0.6 |
| | 9 | 4.82 ± 0.06 | 69 | 1.43 ± 0.12b | 66.7 ± 0.7a | 48 ± 4b | −17.9 ± 0.9 |

*Data are expressed in mean ± SD (n = 3)
**Data are expressed in mean ± SD (n = 3). Significant differences within PG group or WCS group are denoted by different letters (p < 0.05).

Molecular Weight, Radius of Gyration, Dispersed Molecular Density, and Zeta-Potential

The content of amylose in WCS is negligible due to the deficiency of granule-bond starch synthase. Therefore, the dispersion of WCS is a dispersion of amylopectin, a branched α-D-glucan. As shown in Table 1, the weight-average molecular weight (M_{w}) of PG (3.11×10⁷ g/mol) was higher than that of WCS (2.79×10⁷ g/mol). However, the z-average radius of gyration (R_{z}) value of PG (30.5 nm) was much lower than that of WCS (60.5 nm), which resulted in a large difference of dispersed molecular density (ρ) in aqueous solution. The ρ value for PG was 1095 g/mol·nm³, compared to 127 g/mol·nm³ for WCS.

Substitution with OS groups had no significant effect on the M_{w} of PG. Native PG had a M_{w} of 3.11×10⁷ g/mol, the M_{w} for PG-OS with DS 0.0146 and 0.0475 were 2.94 and 3.14×10⁷ g/mol, respectively. In contrast, OS substitution reduces the M_{w} of WCS. For native WCS, M_{w} was 2.79×10⁷ g/mol. For WCS-OS with DS 0.0152 and 0.0482, M_{w} was reduced to 1.41 and 1.43×10⁷ g/mol, respectively. The mechanism of molecular degradation associated with OS substitution of WCS is unknown. However, it is possible that the high pH value (8.5-9.0) encountered during reaction led to minor hydrolysis of glucosidic linkages. PG was more resistant to degradation than amylopectin, suggesting its higher structural integrity than amylopectin.

It appears that the OS substitution only slightly changed R_{z} of PG and WCS (Table 1). At pH 6.8 (pH value of the mobile phase of HPSEC-MALLS-RI), OS groups were negatively charged, which resulted in repulsion among neighboring chain segments. The structural integrity of PG-OS restricts the stretching of outer chains and limits the expansion of the nanoparticles, as evidenced by an almost negligible change of R_{z} at lower DS and minor R_{z} increase at higher DS. For WCS-OS, the flexibility and stretching of individual chains retained the R_{z} value even after a substantial decrease of M_{w} from WCS to WCS-OS.

OS substitution affected the dispersed molecular density (ρ) of glucan molecules (Table 1). The ρ value was reduced from 1095 g/mol·nm³ for native PG to 1036 and 824 g/mol·nm³ for PG-OS with DS 0.0146 and 0.0475, respectively. Apparently, an increased DS led to reduced density, which is attributed to the slightly increased R_{z}. For WCS, the ρ value was substantially reduced from 127 g/mol·nm³ for native WCS to 56 and 48 g/mol·nm³ for WCS-OS with DS 0.0152 and 0.0482, respectively. Evidently, OS substitution increased the difference of molecular density between PG and WCS. The ρ ratio between PG and WCS was increased from 8.6 for native glucans to 18.5 and 17.2 for DS around 0.015 and 0.048, respectively.

Zeta-potential values of PG, WCS, PG-OS, and WCS-OS are shown in Table 1. Higher DS led to higher zeta-potential, suggesting that higher amount of OS groups resulted in higher surface charge density. At equivalent DS, zeta-potential of PG-OS was slightly higher than that of WCS-OS, which might be related to the difference of surface morphology or charge distribution. More studies remain to be conducted to address this issue.

Lipid Oxidation of Emulsions without EPL

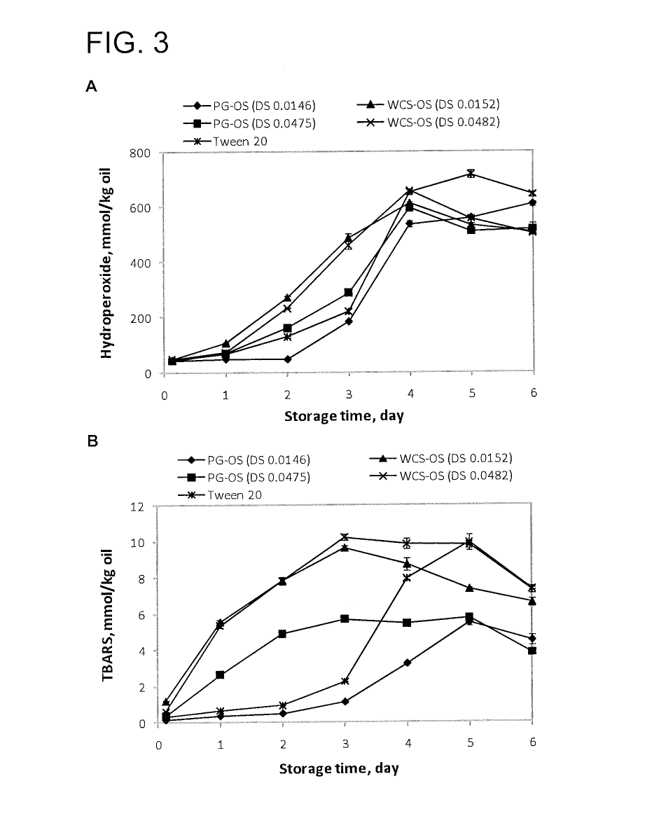

Hydroperoxide value reflects the formation of intermediate compounds of lipid oxidation. As shown in FIG. 3A, the accumulation of lipid hydroperoxide was increased during storage regardless of the use of emulsifiers. The highest values for individual emulsifiers were similar (600-700 mmol/kg oil) and were reached after the fourth day of storage at 55° C. However, the initial hydroperoxide accumulation showed large differences among various emulsifiers, with the emulsion formed by PG-OS (DS 0.0146) having the lowest value before the fourth day, and in particular, before the second day. However, for the emulsion formed using PG-OS (DS 0.0475), hydroperoxide accumulation was much higher in the first 3 days, suggesting that an enhanced DS did not lead to reduced formation of hydroperoxide. Emulsions formed using WCS-OS had higher accumulation of hydroperoxide during the first four days than those using PG-OS. In addition, higher DS for WCS-OS led to slightly lower hydroperoxide at the early stages. In general, PG-OS materials were superior to WCS-OS in retarding the formation of hydroperoxide. Meanwhile, it is shown that the effect of TWEEN 20 ranged between those of two PG-OS materials and was inferior to PG-OS (DS 0.0146) all through the storage period.

TBARS value reflects the formation of final products of lipid oxidation. As shown in FIG. 3B, TBARS of the emulsion made with PG-OS (DS 0.0146) was much lower than that of other emulsifiers during the first 4 days of storage. At the fifth and sixth day, it approached and slightly surpassed the value of PG-OS (DS 0.0475). Similarly to hydroperoxide accumulation, PG-OS (DS 0.0475) was inferior to PG-OS (DS 0.0146) for controlling TBARS, and both PG-OS materials were much more effective than WCS-OS materials. The effect of TWEEN 20 on TBARS accumulation ranged between those of two PG-OS materials, similar to the effect on hydroperoxide accumulation. Among all emulsifiers studied, PG-OS (DS 0.0146) showed the highest capability to retard the formation of both hydroperoxide and TBARS.

Lipid Oxidation of Emulsions with EPL

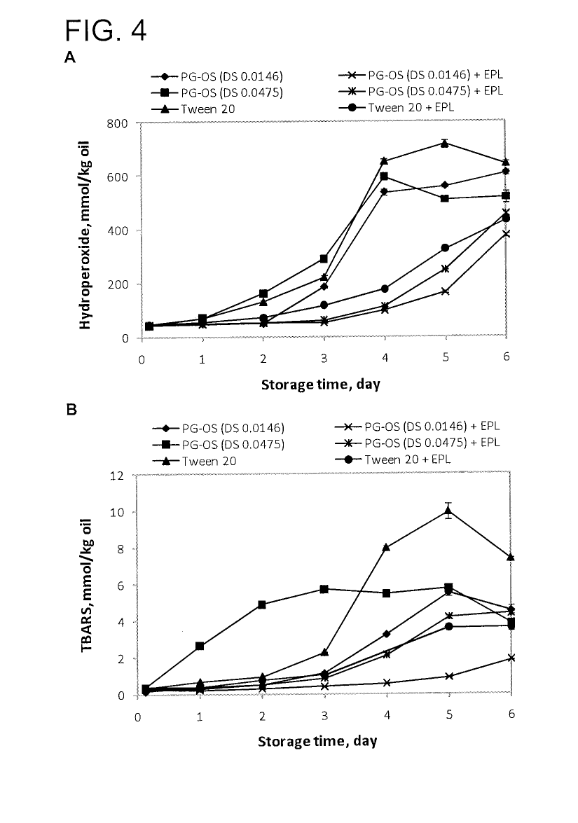

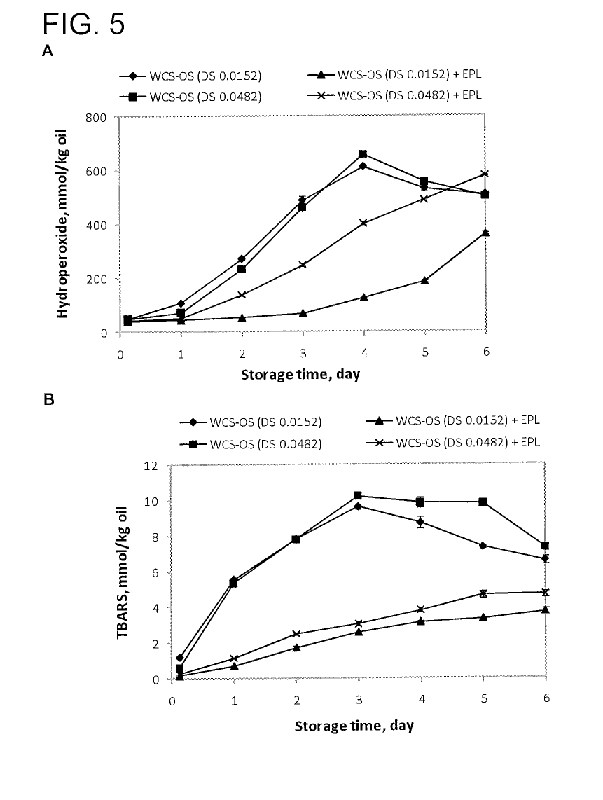

FIGS. 4 and 5 compare the lipid oxidation in the presence or absence of 0.1% EPL. In general, the addition of EPL led to substantial reductions of both hydroperoxide and TBARS for all emulsions. For example, for a PG-OS (DS 0.0146) emulsion, the addition of EPL reduced the hydroperoxide from 536 to 96.3 mmol/kg oil at the fourth day, and from 608 to 374 mmol/kg oil at the sixth day (FIG. 4A). Meanwhile, the TBARS was reduced from 3.2 to 0.6 mmol/kg oil at the fourth day, and from 4.5 to 1.8 mmol/kg oil at the sixth day (FIG. 4B).

For emulsions without EPL, the accumulation of hydroperoxide usually reached the maximum before the sixth day. With EPL, the maximum before the sixth day was not seen for individual emulsions (FIGS. 4A and 5A). Similarly, without EPL the accumulation of TBARS reached the maximum before the sixth day for all emulsions (FIGS. 4B and 5B). In the presence of EPL, at the sixth day the maximum of TBARS was reached for emulsions of PG-OS (DS 0.0475), WCS-OS (DS 0.0482), and TWEEN 20, but not for PG-OS (0.0146) and WCS-OS (DS 0.0152) (FIGS. 4B and 5B).

The effectiveness of EPL to reduce hydroperoxide and TBARS was different when it paired with different emulsifiers. This can be illustrated by comparing PG-OS (DS 0.0475) and TWEEN 20 at the third day. Due to the addition of EPL, hydroperoxide of PG-OS (DS 0.0475) was reduced by 227 mmol/kg oil (from 288 to 61 mmol/kg oil), which was much larger than the reduction of 104 mmol/kg oil for TWEEN 20 (from 220 to 116 mmol/kg oil) (FIG. 4A). Similarly, at the third day, the addition of EPL led to a reduction of 4.9 mmol/kg oil of TBARS (from 5.7 to 0.8 mmol/kg oil) for PG-OS (DS 0.0475), much higher than the reduction of 1.2 mmol/kg oil (from 2.2 to 1.0 mmol/kg oil) for TWEEN 20 (FIG. 4B). Conceivably, the anti-oxidative effect of EPL was associated with its interactions with emulsifiers.

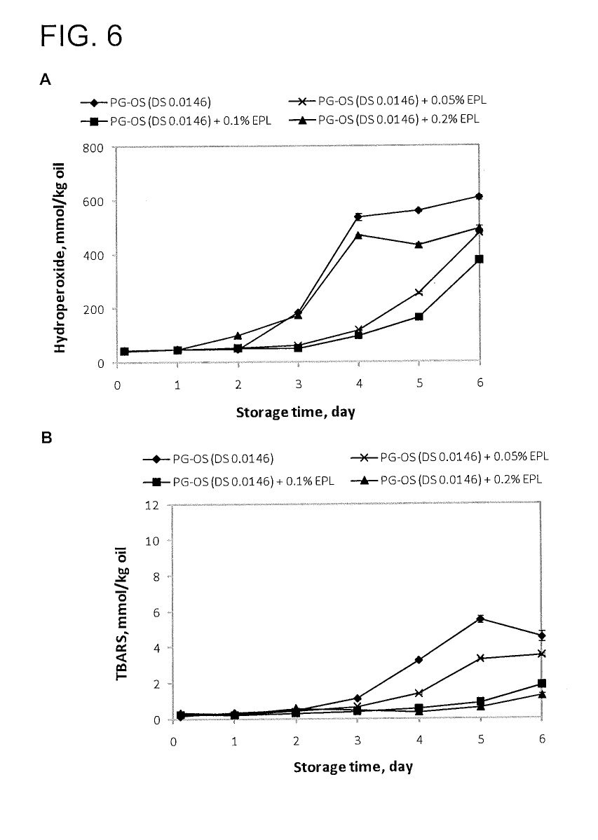

The amount of EPL in emulsion had appreciable effect on lipid oxidation. In FIG. 6, comparisons were made among the use of 0.05, 0.1, and 0.2% of EPL, each paired with 5% PG-OS (DS 0.0146). In terms of reducing hydroperoxide, the effect of 0.05% EPL was slightly lower than that of 0.1% EPL. However, the use of 0.2% EPL led to hydroperoxide accumulation as high as that without EPL (FIG. 6A). For TBARS accumulation, 0.2% EPL appeared to be more effective than 0.1% EPL, and TBARS of 0.05% EPL fall between those of 0.1% and no EPL (FIG. 6B). It is considered that the high hydroperoxide accumulation for 0.2% EPL might be associated with low conversion of hydroperoxide to TBARS.

Particle Size of Emulsions During Storage

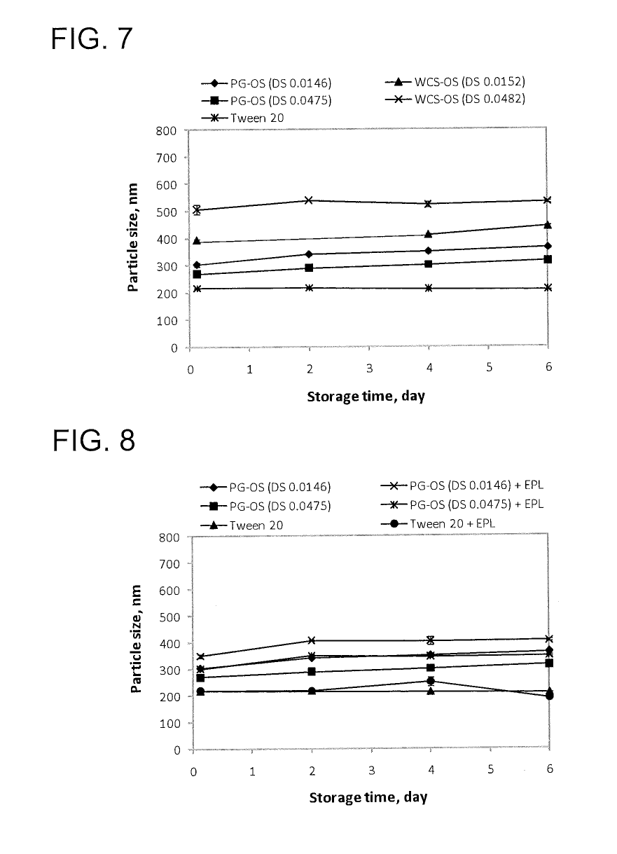

As shown in FIG. 7, the particle size of emulsions was affected by emulsifiers. For example, the initial emulsion particle size of was 216, 303, 269, 393, and 507 nm for TWEEN 20, PG-OS (DS 0.0146), PG-OS (DS 0.0475), WCS-OS (DS-0.0152), and WCS (DS 0.0482), respectively. For PG-OS-stabilized oil droplets, the approximate thickness of interfacial layer can be calculated using R_{z} of nanoparticles (Table 1). Therefore, the actual diameter of oil droplets stabilized using PG-OS (DS 0.0146) and PG-OS (DS 0.0475) should be around 181 nm (303 nm−30.5×2×2 nm) and 134 nm (269 nm−33.7×2×2 nm), respectively. These oil droplets should be smaller than those stabilized using TWEEN 20, since a layer of TWEEN 20 (molecular weight 1227 g/mol) should have very low contribution to the size of oil droplets (216 nm). In contrast, WCS-OS molecules would be more flexible than PG-OS due to its low molecular density. Therefore, it would be more difficult to evaluate the thickness of interfacial layer even though it might be in the scale of dozens of nanometers.

The physical stability of emulsion was slightly different among individual emulsifiers (FIG. 7). After 6 days of storage at 55° C., the particle size was 209, 363, 314, 441, and 532 nm for TWEEN 20, PG-OS (DS 0.0146), PG-OS (DS 0.0475), WCS-OS (DS-0.0152), and WCS (DS 0.0482), respectively. Apparently, TWEEN 20 had the highest capability to maintain the particle size of emulsion. Meanwhile, the increase of particle size was comparable between PG-OS and WCS-OS. In general, all the emulsions showed high physical stability during storage.

As shown in FIG. 8, the addition of 0.1% EPL did not affect the physical stability of emulsions formed using TWEEN 20 and PG-OS. The reason for the fluctuation of particle size for TWEEN 20 emulsion remains unknown. Perhaps it was related to the interaction of EPL and TWEEN 20 at the interface. For both PG-OS emulsifiers, the presence of EPL increased the initial particle size by about 30-50 nm, which was retained all through the storage.

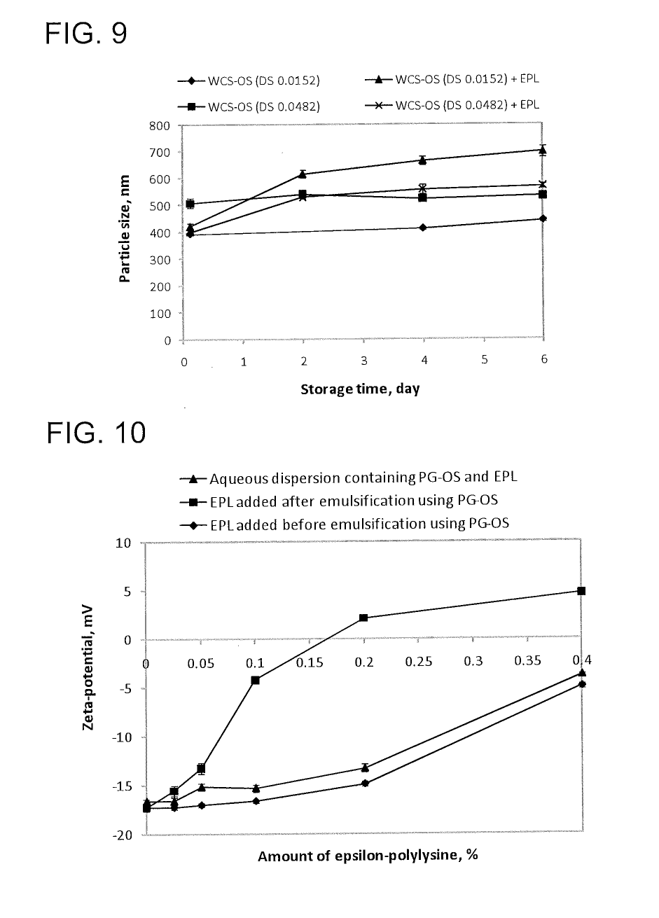

However, for emulsions made using WCS-OS, the addition of EPL led to a substantial increase of particle size during the 6-day storage (FIG. 9). The particle size increased from 422 to 697 nm for WCS-OS (DS 0.0152) and from 399 to 568 nm for WCS-OS (DS 0.0482). Evidently, the physical stability of emulsions containing EPL was strongly affected by the structural differences between PG-OS and WCS-OS.

Zeta-Potential of Systems Containing PG-OS and EPL

Zeta-potential is the electric potential between the slipping plane (within the interfacial double layer) and the bulk fluid away from the interface. It is a very useful parameter for evaluating the stability of colloidal dispersion and the interactions among charged molecules. In this study, zeta-potential was used to understand the interactions between EPL molecules and PG-OS nanoparticles in emulsions and aqueous dispersions.

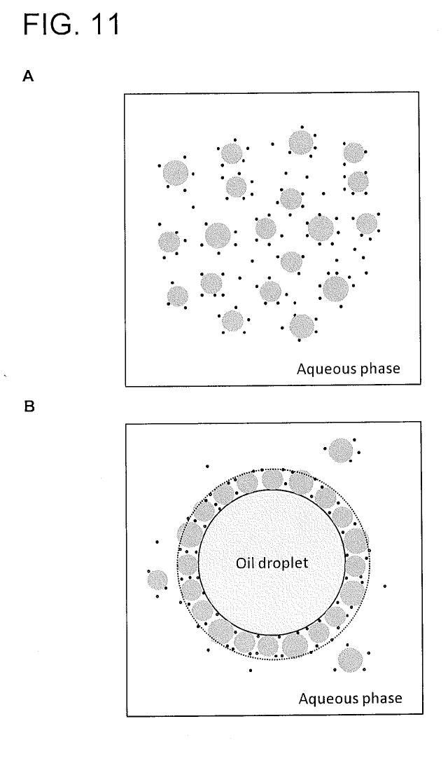

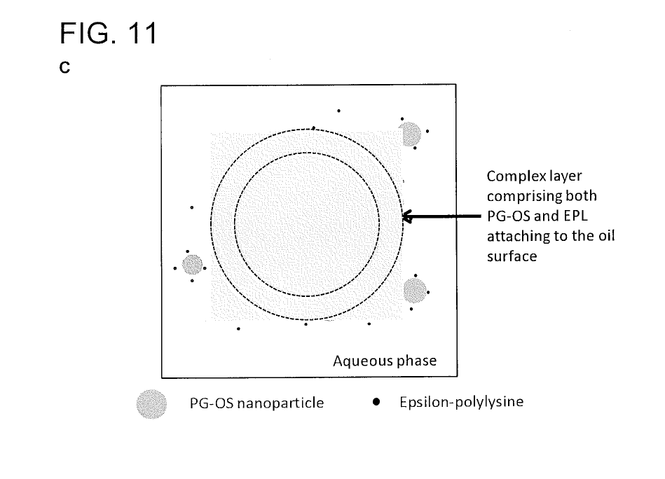

FIG. 10 shows the zeta-potential of systems containing 5% PG-OS and 0, 0.025, 0.05, 0.1, 0.2, and 0.4% EPL for: (1) aqueous dispersions, (2) emulsions with EPL added before emulsification, and (3) emulsions with EPL added after emulsification. For the aqueous dispersions, when EPL was increased from 0 to 0.2%, the zeta-potential of PG-OS slightly changed from −16.63 mV to −13.30 mV. When EPL reached 0.4%, zeta-potential changed to −3.77 mV. The result showed that below 0.2%, the amount of EPL molecules at the slipping plane was not sufficient to cause a substantial change of zeta-potential. At a rather high content (0.4%), the partitioning of EPL molecules at the surface of nanoparticles increased, thereby leading to a large reduction of zeta-potential. FIG. 11A depicts the distribution of EPL in the PG-OS dispersion. In the schematic, there is an enrichment of EPL molecules in the vicinity of PG-OS nanoparticles. However, the portion of EPL molecules distributed at the slipping plane (shown as the boundary of particles) is rather low.

For the groups with EPL added after emulsification, the impact of EPL amount on zeta-potential was different from that for the aqueous dispersions. As shown in FIG. 10, when EPL amount was increased from 0 to 2%, the zeta-potential was quickly changed from −17.23 mV to +2.10 mV, suggesting an effective neutralization of charged colloidal surface. FIG. 11B depicts the distribution of EPL molecules in a PG-OS emulsion. At the oil-water interface, negatively charged PG-OS nanoparticles repel each other and form spaces among neighboring nanoparticles. EPL molecules added into PG-OS emulsion migrate toward oil-water interface under two forces: (1) electrostatic interaction between positively charged EPL amine groups and negatively charged PG-OS carboxylate groups, and (2) hydrophobicity of EPL molecules. At equilibrium (or possibly quasi-equilibrium), a portion of EPL molecules filled in the spaces formed by neighboring PG-OS nanoparticles. These EPL molecules could effectively neutralize the negative charge at the slipping plane of PG-OS interfacial layer (indicated by the dotted circle). When EPL amount increases, the filling of EPL molecules effectively increases, causing rapid change of zeta-potential.

When EPL was added before emulsification, the increase of EPL resulted in minor increase of zeta-potential (FIG. 10). When EPL increased from 0 to 0.2%, zeta-potential changed from −17.23 mV to −14.90 mV. When EPL reached 0.4%, zeta-potential was changed to −4.94 mV. This information is useful for evaluating the position of EPL molecules in emulsion. Without wishing to be bound by a particular theory or to in any way limit the scope of the appended claims or their equivalents, it is presently believed that most EPL molecules were not in the bulk of aqueous phase since otherwise the system would be unstable and quickly transfer to the same state as that with EPL added after emulsification. It is highly possible that most EPL molecules were adsorbed at the oil surface, rather than at the outer surface of PG-OS interfacial layer (FIG. 11C). In this scenario, there could be a well-defined complex layer formed at the oil-water interface (FIG. 11C). In the complex layer, both EPL molecules and PG-OS nanoparticles directly contact with oil phase. Due to the large size of PG-OS nanoparticles, the surface charge of entire droplet was governed by the negatively charged OS carboxylate groups.

Interfacial Complex Layer of Amphiphilic Nanoparticles and EPL

EPL is amphiphilic, which allows for its interfacial enrichment even though it is a weak emulsifier. A stable EPL adsorption at the surface of oil droplets requires the formation of stable oil droplets and available adsorption sites. Stable oil droplets can be formed using regular emulsifiers. However, small-molecule emulsifiers (e.g., TWEEN 20) and structurally flexible polymeric emulsifiers (e.g., WCS-OS) may occupy a large portion of oil surface. In contrast, amphiphilic nanoparticles can be suitable emulsifiers to form stable oil droplets while providing available adsorption sites for EPL. The nanoparticles should be rigid enough to avoid excess coverage of adsorption sites by flexible chains.

Due to its structural integrity, PG-OS is a suitable material for forming stable oil droplets and providing adsorption sites for EPL. Without wishing to be bound by a particular theory or to in any way limit the scope of the appended claims or their equivalents, it is presently believed that that the interfacial PG-OS/EPL complex layer has two basic functions: (1) being thick and dense, thus forming a strong physical barrier for the permeation of pro-oxidative compounds (e.g. oxygen and metal ions), and (2) being positively charged at the surface of oil droplets due to adsorbed EPL molecules, thus repelling metal ions. Therefore, a combined use of PG-OS and EPL effectively blocked the permeation of pro-oxidative compounds and resulted in high lipid oxidative stability of emulsions.

The following literature provides information that may be useful in accordance with the present teachings and each document is hereby incorporated by reference in its entirety, except that in the event of any inconsistent disclosure or definition from the present specification, the disclosure or definition herein shall be deemed to prevail: (1) James, M.; Robertson, D.; Myers, A. “Characterization of the maize gene sugary1, a determinant of starch composition in kernels,” Plant Cell, 1995, 7, 417-429; (2) Yao, Y. Chapter: Biosynthesis of starch, in Comprehensive Glycoscience, edited by Hans Kamerling, Elsevier, 2007; (3) Myers, A.; Morell, M.; James, M.; Ball, S. “Recent progress toward understanding the amylopectin crystal,” Plant Physiology, 2000, 122, 989-997; (4) Nakamura, Y. “Towards a better understanding of the metabolic system for amylopectin biosynthesis in plants:

Rice endosperm as a model tissue,” Plant and Cell Physiology, 2002, 43, 718-725; (5) Putaux, J.; Buleon, A.; Borsali R.; Chanzy, H. “Ultrastructural aspects of phytoglycogen from cryo-transmission electron microscopy and quasi-elastic light scattering data,” International Journal of Biological Macromolecules, 1999, 26, 145-150; (6) Wong, K.; Kubo, A.; Jane, J.; Harada, K.; Satoh, H.; Nakamura, Y. “Structures and properties of amylopectin and phytoglycogen in the endosperm of sugary-1 mutants of rice,” Journal of Cereal Science, 2003, 37, 139-149; (7) Thompson, D. B. “On the non-random nature of amylopectin branching,” Carbohydrate Polymer, 2000, 43, 223-239; (8) Shin, J.; Simsek, S.; Reuhs, B.; Yao Y. “Glucose release of water-soluble starch-related α-glucans by pancreatin and amyloglucosidase is affected by the abundance of α-1,6 glucosidic linkages,” Journal of Agricultural and Food Chemistry, 2008, 56, 10879-10886; (9) Caldwell, C. G.; Wurzburg, O. B. “Polysaccharide derivatives of substituted dicarboxylic acids,” U.S. Pat. No. 2,661,349; (10) Shogren, R. L.; Viswanathan, A.; Felker, F.; Gross, R. A. “Distribution of octenyl succinate groups in octenyl succinic anhydride modified waxy maize starch,” Starch-Stärke, 2000, 52, 196-204; (11) Bhosale, R.; Singhal, R. “Effect of octenyl succinylation on physicochemical and functional properties of waxy maize and amaranth starches,” Carbohydrate Polymers, 2007, 68, 447-456; (12) Song, X. Y.; He, G. Q.; Ruan, H.; Chen, Q. H. “Preparation and properties of octenyl succinic anhydride modified early indica rice starch,” Starch-Starke, 2006, 58, 109-117; (13) Liu, Z. Q.; Li, Y.; Cui, F. J.; Ping, L. F.; Song, J. M.; Ravee, Y.; Jin, L. Q.; Xue, Y. P.; Xu, J. M.; Li, G.; Wang, Y. J.; Zheng, Y. G. “Production of octenyl succinic anhydride-modified waxy corn starch and its characterization,” Journal of Agricultural and Food Chemistry, 2008, 56, 11499-11506; (14) Drusch, S.; Serfert, Y.; Scampicchio, M.; Schmidt-Hansberg, B.; Schwarz, K. “Impact of physicochemical characteristics on the oxidative stability of fish oil microencapsulated by spray-drying,” Journal of Agricultural and Food Chemistry, 2007, 55, 11044-11051; (15) Shih, I. L.; Shen, M. H.; Van, Y. T. “Microbial synthesis of poly (ε-lysine) and its various applications,” Bioresource Technology, 2006, 97, 1148-1159; (16) Nawar, W. W. “Lipids” in Food chemistry, 3rd edition, edited by Fennema, O. R., Marcel Dekker, New York, 1996; (17) Decker, E. A. “Strategies for manipulating the pro-oxidative/antioxidative balance of foods to maximize oxidative stability,” Trends in Food Science and Technology, 1998, 9, 241-248; (18) McClements, D. J.; Decker, E. A. “Lipid oxidation in oil-in-water emulsions: impact of molecular environment on chemical reactions in heterogeneous food systems,” Journal of Food Science, 2000, 65, 1270-1282; (19) Ogawa, S.; Decker, E. A.; McClements, D. J. “Influence of environmental conditions on the stability of oil in water emulsions containing droplets stabilized by lecithin-chitosan membranes,” Journal of Agricultural and Food Chemistry, 2003, 51, 5522-5527; (20) Klinkesorn, U.; Sophanodora, P.; Chinachoti, P.; Mcclements, D. J.; Decker, E. A. “Increasing the oxidative stability of liquid and dried tuna oil-in-water emulsions with electrostatic layer-by-layer deposition technology,” Journal of Agricultural and Food Chemistry, 2005, 53, 4561-4566; (21) Guzey, D.; McClements, D. J. “Formation, stability and properties of multilayer emulsions for application in the food industry,” Advances in Colloid and Interface Science, 2006, 128, 227-248; (22) Katsuda, M. S.; Mcclements, D. J.; Miglioranza, L. H. S.; Decker, E. A. “Physical and oxidative stability of fish oil-in-water emulsions stabilized with beta-lactoglobulin and pectin,” Journal of Agricultural and Food Chemistry, 2008, 56, 5926-5931; (23) Kellerby, S. S; Gu, Y. S.; McClements, D. J.; Decker, E. A. “Lipid oxidation in a menhaden oil-in-water emulsion stabilized by sodium caseinate cross-linked with transglutaminase,” Journal of Agricultural and Food Chemistry, 2006, 54, 10222-10227; (24) DS titration method CXAS. Joint FAO/WHO Expert Committee on Food Additives (JECFA), 1991, page 985; (25) Pilz, J.; Meineke, I.; Gleiter, C. H. “Measurement of free and bound malondialdehyde in plasma by high-performance liquid chromatography as the 2,4-dinitrophenylhydrazine derivative,” Journal of Chromatography B-Analytical Technologies in the Biomedical and Life Sciences, 2000, 742, 315-325; (26) Ho, Y.; Ishizaki, S.; Tanaka, M. “Improving emulsifying activity of ε-polylysine by conjugation with dextran through the Maillard reaction,” Food Chemistry, 2000, 68, 449-455; and (27) Scheffler, S. L.; Wang, X.; Huang, L.; San-Martin Gonzalez, F.; Yao, Y. “Phytoglycogen Octenyl Succinate, an Amphiphilic Carbohydrate Nanoparticle, and e-Polylysine to Improve Lipid Oxidative Stability of Emulsions,” J. Agric. Food Chem., 2010, 58, 660-667.

The foregoing detailed description and accompanying drawings have been provided by way of explanation and illustration, and are not intended to limit the scope of the appended claims. Many variations in the presently preferred embodiments illustrated herein will be apparent to one of ordinary skill in the art, and remain within the scope of the appended claims and their equivalents.

1. An emulsion comprising:
a lipid;
an emulsifier; and
ε-polylysine.

2. The invention of claim 1 wherein the lipid comprises an omega-3 fatty acid and/or an ester derivative thereof.

3. The invention of claim 2 wherein the omega-3 fatty acid and/or the ester derivative are selected from the group consisting of fish oil, an ester derivative of fish oil, docosahexaenoic acid, an ester derivative of docosahexaenoic acid, eicosapentaenoic acid, an ester derivative of eicosapentaenoic acid, and combinations thereof.

4. The invention of claim 1 wherein the emulsifier comprises a food grade emulsifier.

5. The invention of claim 1 wherein the emulsifier is selected from the group consisting of amphiphilic proteins, phospholipids, small molecule surfactants, polysorbate surfactants, gum arabic, modified starch, modified phytoglycogen, modified glycogen-type materials, and combinations thereof.

6. The invention of claim 1 wherein the emulsifier is selected from the group consisting of gum arabic, polysorbate 20, anhydride-modified starch, anhydride-modified phytoglycogen, anhydride-modified glycogen-type material, and combinations thereof.

7. The invention of claim 6 wherein the anhydride comprises succinic anhydride.

8. The invention of claim 6 wherein the anhydride comprises octenyl succinic anhydride.

9. The invention of claim 1 wherein the emulsifier comprises phytoglycogen octenyl succinate.

10. The invention of claim 9 wherein a degree of substitution of the phytoglycogen octenyl succinate is between about 0.002 and about 1.00.

11. The invention of claim 9 wherein a degree of substitution of the phytoglycogen octenyl succinate is between about 0.010 and about 0.050.

12. The invention of claim 9 wherein a degree of substitution of the phytoglycogen octenyl succinate is about 0.015.

13. The invention of claim 9 wherein a degree of substitution of the phytoglycogen octenyl succinate is about 0.048.

14. The invention of claim 1 wherein the ε-polylysine comprises from about 0.01% to about 1.0% by weight of the emulsion.

15. The invention of claim 1 wherein the ε-polylysine comprises about 0.1% by weight of the emulsion.

16. An emulsion comprising:
a lipid;
an emulsifier selected from the group consisting of anhydride-modified phytoglycogen, anhydride-modified glycogen-type material, and combinations thereof; and
ε-polylysine.

17. A method for improving oxidative stability of a lipid comprising:
forming an emulsion comprising the lipid, an emulsifier, and ε-polylysine;
wherein the emulsion further comprises a complex layer at an oil-water interface configured to provide a physical and/or electrostatic barrier against oxidation of the lipid.

18. The invention of claim 17 wherein the emulsifier is selected from the group consisting of anhydride-modified phytoglycogen, anhydride-modified glycogen-type material, anhydride-modified starch, and combinations thereof.

19. The invention of claim 17 wherein the emulsifier comprises phytoglycogen octenyl succinate.

20. A method for preparing an emulsion comprising:
combining a lipid, an emulsifier, and ε-polylysine in a mixture; and
homogenizing the mixture to provide an emulsion;
wherein the emulsifier is selected from the group consisting of anhydride-modified phytoglycogen, anhydride-modified glycogen-type material, anhydride-modified starch, and combinations thereof.

21. The invention of claim 20 wherein the mixture further comprises water.

22. The invention of claim 20 wherein the mixture further comprises a buffer.

23. The method of claim 20 wherein the emulsifier comprises phytoglycogen octenyl succinate.
